(19) United States
(12) Patent Application Publication
(10) Pub. No.: US 2026/0130859 A1
(43) Pub. Date: May 14, 2026

(54) PALATABLE FORMULATIONS

(71) Applicant: Elanco US Inc., Greenfield, IN (US)

(72) Inventor: Rajesh PATIL, Greenfield, IN (US)

(21) Appl. No.: 19/443,861

(22) Filed: Jan. 8, 2026

Related U.S. Application Data
(62) Division of application No. 18/043,743, now Pat. No. 12,544,336.
(60) Provisional application No. 63/075,055, filed on Sep. 4, 2020.

(51) Int. Cl. A61K 9/20; A61K 9/00; A61K 31/365; A61K 31/422; A61K 31/4985

(52) U.S. Cl. CPC A61K 9/2081; A61K 9/0056; A61K 31/365; A61K 31/422; A61K 31/4985

(57) ABSTRACT

Palatable oral dosage formulations are provided including an effective amount of an isoxazoline parasiticidal agent, an avermectin, and a pyrazinoisoquinoline, and optionally one or more additional active ingredients, such as a tetrahydropyrimidine.

### CROSS-REFERENCE TO RELATED APPLICATIONS

This application is a divisional of U.S. patent application Ser. No. 18/043,743, filed Mar. 2, 2023, which is a National Stage entry of International Application No. PCT/US2021/048842, filed Sep. 2, 2021, which claims priority to U.S. Provisional Patent Application No. 63/075,055, filed Sep. 4, 2020, the entire contents of each of which are incorporated herein by reference.

### BACKGROUND

### Technical Field

The present disclosure relates to palatable veterinary compositions, processes for preparing the same, and to methods of using the same, for example, for the treatment of parasitic infections.

### Description of Related Art

Ectoparasites such as fleas, lice, flies, mosquitoes, ticks and mites, as well as endoparasites such as gastrointestinal tract nematodes, flukes, and filaroids, are problematic for man and animal alike. Such parasites seriously impact productivity in the domesticated animal industry by reducing weight gain, causing poor quality hide, wool, and meat, and in some cases resulting in death. Parasites are also responsible, in part, for the spread of disease and discomfort in mammals, including food and companion animals. Ectoparasites in particular are known to harbor and transmit a variety of microbial pathogens, including bacteria, viruses and protozoan parasites, many of which are pathogenic to humans, other warm-blooded mammals, and birds. Ectoparasites have been implicated in a number of diseases including, for example, malaria, lymphatic- and blood-born filariasis, trachoma, trypanosomiasis, Leishmaniasis, Rocky Mountain Spotted Fever, Lyme Disease, babesiosis, and food-borne illnesses due to Salmonella, E. coli and Campylobacter, as well as others.

The significant health implications of parasitic infestations have boosted the development of reagents capable of controlling parasites and parasitic infestations in plants and animals.

Common methods for controlling parasitic infestation generally focus on methods that incorporate one or more insecticides, which in many situations may prove unsuccessful or unsatisfactory for any number of reasons including, for example, (1) failure of administration compliance (frequent administration by owner required); (2) behavioral or physiological intolerance of the animal to the pesticide product or means of administration; (3) the emergence of ectoparasites resistant to the reagent; and (4) negative impact on the environment and/or toxicity.

In the field of veterinary medicine, oral administration of medicaments is desirable because it can often be carried out without the involvement of a veterinarian. However, creating oral veterinary products incorporating multiple active agents requires consideration of several variables, including, drug solubility, drug bioavailability, palatability, active agent stability, toxicity, safety, efficacy (potency and duration), resistance, issues relating to any side effect(s), and patient/owner compliance with the administration schedule.

WO/2002/045693 A1 discloses a preparation with an active ingredient essentially uniformly dispersed in an excipient matrix composed of one or more excipients selected from the group of fatty alcohol, triglyceride, partial glyceride and fatty acid ester. WO/2003/075895 A1 discloses an animal medicine in an application form, which contains the active ingredient in a stabilized form that masks the taste, and which is readily taken orally by an animal. WO/2005/079759A2 discloses a pharmaceutical composition for oral application comprising: i) an anthelmintic agent; ii) a first excipient having a porous structure with an inner surface of about 500 to 1500 m2/g and a surface area according to BET of up to about 5000 m2/g. WO/2006/036624A2 discloses a lipid-based system for isolating components in a pharmaceutical formulation.

WO/2006/036625A2 discloses lipid-based pharmaceutical formulations containing ivermectin, praziquantel and pyrantel pamoate, and methods for preparing the formulations.

WO/2008/148484 A1 discloses extrudates which contain one or more pharmaceutically active substances and have a maximum bar diameter of 0.5 mm. WO/2009/023013A1 discloses a multiparticulate in-feed additive composition comprising praziquantel or a salt thereof and cimetidine or a salt thereof. WO/2010/063387A1 discloses extrudates comprising at least one pharmaceutically active substance in the shape of pins, wherein the ratio of particle sizes of the spicular pharmaceutically active substance to the strand diameter is at least 1:15.

WO/2020/051106A1 discloses a palatable, hard chewable composition that comprises at least one veterinary acceptable isoxazoline, a stabilized macrocyclic lactone, an acceptable salt form of pyrantel, at least one natural animal based palatant, and at least one veterinary acceptable excipient.

### BRIEF DESCRIPTION OF THE DRAWINGS

FIG. 1 shows an exemplary embodiment to produce the disclosed compositions.

### DETAILED DESCRIPTION

This disclosure provides oral dosage formulations comprising an effective amount of an isoxazoline parasiticidal agent, an avermectin, and a pyrazinoisoquinoline, and optionally one or more additional active ingredients, such as a tetrahydropyrimidine. The formulations and methods not only provide alternatives to those that currently exist in the art, but also overcome one or more limitations associated with current formulations such as, drug solubility, drug bioavailability, palatability, active agent stability, toxicity, safety, efficacy (potency and duration), spectrum of parasiticidal activity, resistance, issues relating to any side effect(s), or patient/owner compliance with administration.

In one aspect, the present disclosure provides oral dosage formulations including an isoxazoline, moxidectin, and praziquantel, and optionally pyrantel or a salt thereof. Praziquantel is a bitter and obnoxious smelling active, which can lead to the rejection of the dosage form by a patient. Moxidectin is a labile active, sensitive to exposure to acid, alkali, light, and oxidizing conditions, and thus can present stability issues when incorporating it into an oral dosage formulation.

Furthermore, both actives are poorly soluble in water, creating challenges for bioavailability.

It has been discovered that praziquantel can be successfully taste/odor-masked by confining the praziquantel to discrete areas within the matrix of the oral dosage formulation. This is accomplished by preparing a mixture of praziquantel granules to provide a first granulate; optionally coating the first granulate with a physiologically acceptable polymer matrix (e.g., polymethacrylate-based copolymer matrix); preparing granules of one or more additional actives to provide a second granulate; combining the first and second granulates and at least one pharmaceutically acceptable excipient to provide a third blend (which may also be referred to as the “third granulate”); and compressing this blend (the “final mixture” or “ready-to-press mixture”) into a tablet.

It has further been discovered that co-granulating moxidectin with the praziquantel provides a granulate in which moxidectin is sufficiently stable for the present purposes. This is accomplished by preparing a mixture of praziquantel and moxidectin granules to provide a first granulate; optionally coating the first granulate with a physiologically acceptable polymer matrix (e.g., polymethacrylate-based copolymer matrix); preparing granules of an isoxazoline and optionally one or more additional actives to provide a second granulate; combining the first and second granulates and at least one pharmaceutically acceptable excipient to provide a third blend; and compressing the ready-to-press mixture into a tablet.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term, “administering to a subject” includes but is not limited to cutaneous, subcutaneous, intramuscular, mucosal, submucosal, transdermal, oral or intranasal administration.

Administration could include injection or topical administration.

The term, “chewable” refers to a solid form, which can be taken by mouth and crushed into smaller pieces before swallowing.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “salt” refers to salts of veterinary or pharmaceutically acceptable organic acids and bases or inorganic acids and bases. Such salts are well known in the art and include those described in Journal of Pharmaceutical Science, 66, 2-19 (1977).

The skilled artisan will appreciate that certain of the compounds of the present invention exist as isomers. All stereoisomers of the compounds of the invention, including geometric isomers, enantiomers, and diastereomers, in any ratio, are contemplated to be within the scope of the present invention.

The skilled artisan will also appreciate that certain of the compounds of the present invention exist as tautomers. All tautomeric forms the compounds of the invention are contemplated to be within the scope of the present invention.

Compounds of the invention also include all isotopic variations, in which at least one atom of the predominant atom mass is replaced by an atom having the same atomic number, but an atomic mass different from the predominant atomic mass. Use of isotopic variations (e.g., deuterium, ²H) may afford greater metabolic stability. Additionally, certain isotopic variations of the compounds of the invention may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies.

The terms “compounds of the invention” and “a compound of the invention” and “compounds of the present invention” and a like include the chemical compounds, in particular the active ingredients (also referred to as “actives”) described herein and the exemplified compounds described herein and a salt of each of these embodiments.

The terms “treating”, “to treat”, “treated”, or “treatment”, include without limitation restraining, slowing, stopping or reversing the progression or severity of an existing symptom or disease. The terms “control”, “controlling” or “controlled” refers to include without limitation decreasing, reducing, or ameliorating the risk of a symptom, disorder, condition, or disease, and protecting an animal from a symptom, disorder, condition, or disease. Controlling may refer to therapeutic, prophylactic, or preventative administration. For example, a larvae or immature heartworm infection would be controlled by acting on the larvae or immature parasite preventing the infection from progressing to an infection by mature parasites.

The term “preventing” refers to the avoidance of a symptom or disease developing in an animal. The terms “subject” and “patient” refers includes humans and non-human mammalian animals, such as dogs, cats, mice, rats, guinea pigs, rabbits, ferrets, cows, horses, sheep, goats, and pigs. It is understood that a more particular subject is a human. Also, a more particular subject are mammalian pets or companion animals, such as dogs and cats and also mice, guinea pigs, ferrets, and rabbits.

The term “effective amount” refers to an amount which gives the desired benefit to the subject, and includes administration for treatment or control. The amount will vary from one individual subject to another and will depend upon a number of factors, including the overall physical condition of the subject and the severity of the underlying cause of the condition to be treated, concomitant treatments, and the amount of compound of the invention used to maintain desired response at a beneficial level.

An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, infection, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present disclosure, the active ingredient treatment dosage, may range from, for example, 0.5 mg to 100 mg. Specific amounts can be determined by the skilled person. Although these dosages are based on a subject having a mass of about 1 kg to about 20 kg, the diagnostician will be able to determine the appropriate dose for a subject whose mass falls outside of this weight range. An effective amount of the present disclosure, the active ingredient treatment dosage, may range from, for example, 0.1 mg to 10 mg/kg of the subject. The dosing regimen is expected to be daily, weekly, or monthly administration.

The phrases “oral bioavailability” and “bioavailability upon oral administration” as used herein refer to the systemic availability (i.e., blood/plasma levels) of a given amount of active administered to a patient.

The term “clearance” as used herein refers to the removal of a substance from the blood, e.g., by renal excretion, expressed in terms of the volume flow of blood or plasma that would contain the amount of substance removed per unit time.

The term “half-life” as used herein refers to the period of time required for one-half of an amount of a substance to be lost through biological processes.

The term “bioavailability” as used herein refers to the physiological availability of a given amount of a drug, as distinct from its chemical potency. The term may also refer to the proportion of the administered dose which is absorbed into the bloodstream.

The term “animal” is used herein to include all vertebrate animals, including humans, companion animals, and livestock animals. It also includes an individual animal in all stages of development, including embryonic and fetal stages. Companion animals include, but are not limited to, dogs and cats. Livestock animals include, but are not limited to, cattle, camelids, pigs, sheep, goats and horses.

The term “parasite” as used herein refers to a pest which lives in or on the host animal and benefits by deriving nutrients at the host animal's expense. The term encompasses all stages in the lifecycle of the pest. An “endoparasite” is a parasite which lives in the host animal. An “ectoparasite” is a parasite which lives on the host animal.

The term “wt/wt” or “w/w” designates weight/weight, the term “w/v” designates weight/volume, and the term “mg/kg” designates milligrams per kilogram of body weight. The term “% wt/wt” represents the percentage by weight of an ingredient in the recipe of a composition.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITIONS

### Actives

Compositions of the present disclosure include an isoxazoline class parasiticide, an avermectin (e.g., ivermectin, selamectin, doramectin, eprinomectin, moxidectin, and abamectin), and a pyrazinoisoquinoline (e.g., praziquantel). The compositions optionally include one or more additional actives, such as for example, a milbemycin (e.g., milbemycin oxime), a benzimidazole (e.g., fenbendazole, albendazole, and triclabendazole), a salicylanilide (e.g., closantel and oxyclozanide), a substituted phenol (e.g., nitroxynil), a tetrahydropyrimidine (e.g., pyrantel, morantel, oxantel), an imidazothiazole (e.g., levamisole), or a cyclooctadepsipeptide (e.g., emodepside).

The isoxazoline class parasiticide may have the formula:

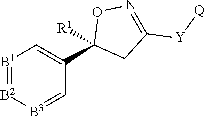

wherein B¹, B², B³, are each independently C—R or N; each R is independently H, halogen, cyano, —NO₂, alkyl, haloalkyl, alkoxy, haloalkoxy, alkylthio, haloalkylthio, alkylsulfinyl, haloalkylsulfinyl, alkylsulfonyl, haloalkylsulfonyl, alkylamino, dialkylamino or alkoxycarbonyl; R¹ is C₁-C₃alkyl or C₁-C₃haloalkyl; Y is an optionally substituted phenylene, naphthylene, indanylene, a 5- or 6-membered heteroarylene or an 8-10-membered fused heterobicyclylene, wherein the optional substituents are selected from the group consisting of halogen, alkyl, haloalkyl, cycloalkyl, halocycloalkyl, alkoxy, haloalkoxy, alkylthio, haloalkylthio, alkylsulfinyl, haloalkylsulfinyl, alkylsulfonyl, haloalkylsulfonyl, alkylamino, dialkylamino, —CN or —NO₂ and NH₂—C(═S)—; Q is T-NR²R³, the group (—CH₂—)(—CH₂—)N—R³, OH, NH₂, alkoxy, haloalkoxy, alkylamino, haloalkylamino, dialkylamino, halodialkylamino, thiol, alkylthio, haloalkylthio, alkylsulfinyl, haloalkylsulfinyl, alkylsulfonyl, haloalkylsulfonyl, or an optionally substituted 5- or 6-membered carbocyclyl, heterocyclyl or heteroaryl ring; T is (CH₂)ₙ, CH(CH₃), CH(CN), C(═O) or C(═S); R² is H, alkyl, alkenyl, alkynyl, cycloalkyl, alkylcycloalkyl, cycloalkylalkyl, alkylcarbonyl or alkoxycarbonyl; R³ is H, OR⁷, NR⁸R⁹ or Q¹; or alkyl, haloalkyl, alkenyl, haloalkenyl, alkynyl, haloalkynyl, cycloalkyl, alkylcycloalkyl, cycloalkylalkyl, alkylcarbonyl, alkoxycarbonyl, aminocarbonyl, alkylaminocarbonyl or dialkylaminocarbonyl, each optionally substituted with one or more substituents independently selected from R⁴; or R² and R³ are taken together with the nitrogen to which they are attached to form a ring containing 2 to 6 atoms of carbon and optionally one additional atom selected from the group consisting of N, S and O, said ring optionally substituted with 1 to 4 substituents independently selected from the group consisting of alkyl, halogen, —CN, —NO₂ and alkoxy; each R⁴ is independently halogen; alkyl, cycloalkyl, alkoxy, alkylthio, haloalkylthio, alkylsulfinyl, haloalkylsulfinyl, alkylsulfonyl, haloalkylsulfonyl, alkylamino, haloalkylamino, dialkylamino, dihaloalkylamino, cycloalkylamino, alkylcarbonyl, alkoxycarbonyl, alkylaminocarbonyl, dialkylaminocarbonyl, haloalkyl carbonyl, haloalkoxycarbonyl, haloalkylaminocarbonyl, dihaloalkylaminocarbonyl, hydroxy, —NH₂, —CN or —NO₂; or Q²; each R⁵ is independently halogen, alkoxy, haloalkoxy, alkylthio, haloalkylthio, alkylsulfinyl, haloalkylsulfinyl, alkylsulfonyl, haloalkylsulfonyl, alkylamino, dialkylamino, alkoxycarbonyl, —CN or —NO₂; each R⁶ is independently halogen, alkyl, haloalkyl, cycloalkyl, halocycloalkyl, alkoxy, haloalkoxy, alkylthio, haloalkylthio, alkylsulfinyl, haloalkylsulfinyl, alkylsulfonyl, haloalkylsulfonyl, alkylamino, dialkylamino, —CN, —NO₂, phenyl or pyridinyl; R⁷ is H; or alkyl, alkenyl, alkynyl, cycloalkyl, alkylcycloalkyl or cycloalkylalkyl, each optionally substituted with one of more halogen; R³ is H, alkyl, alkenyl, alkynyl, cycloalkyl, alkylcycloalkyl, cycloalkylalkyl, alkylcarbonyl or alkoxycarbonyl; R⁹ is H; Q³; or alkyl, alkenyl, alkynyl, cycloalkyl, alkylcycloalkyl or cycloalkylalkyl, each optionally substituted with one or more substituents independently selected from R⁴; or R³ and R⁹ are taken together with the nitrogen to which they are attached to form a ring containing 2 to 6 atoms of carbon and optionally one additional atom selected from the group consisting of N, S and O, said ring optionally substituted with 1 to 4 substituents independently selected from the group consisting of alkyl, halogen, —CN, —NO₂ and alkoxy; Q¹ is a phenyl ring, a 5- or 6-membered heterocyclic ring, or an 8-, 9- or 10-membered fused bicyclic ring system optionally containing one to three heteroatoms selected from up to 10, up to 1 S and up to 3 N, each ring or ring system optionally substituted with one or more substituents independently selected from R⁵; Q² is independently a phenyl ring or a 5- or 6-membered heterocyclic ring, each ring optionally substituted with one or more substituents independently selected from R⁶; Q³ is a phenyl ring or a 5- or 6-membered heterocyclic ring, each ring optionally substituted with one or more substituents independently selected from R⁶; and n is 1, 2 or 3; wherein the asterisk represents that the carbon atom is a quaternary carbon atom. Exemplary isoxazoline class parasiticides include afoxolaner, fluralaner, lotilaner, and sarolaner.

In certain embodiments, the compositions include an insecticide or acaricide. Exemplary insecticides and acaricides include, for example, acephate, acetamiprid, acetoprole, amitraz, amidoflumet, avermectin, azadirachtin, azinphos-methyl, bifenthrin, bifenazate, buprofezin, bistrifluron, buprofezin, carbofuran, cartap, chlorfenapyr, chlorfluazuron, chlorantraniliprole), chlorpyrifos, chlorpyrifos-methyl, chromafenozide, clothianidin, cyflumetofen, cyfluthiin, P-cyfluthrin, cyhalothrin, γ-cyhalothrin k-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim, flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, monocrotophos, nitenpyram, nithiazine, novaluron, noviflumuron, oxamyl, parathion, parathiori-methyl, permethrin, phorate, phosalone, phosmet; phosphamidori, pirimicarb, profenofos, profluthrin, protrifenbute, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen, spirotetramat, sulprofos, tebufenozide, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon, and triflumuron.

In certain embodiments, the compositions include active ingredient(s) ranging from 0.005% to 80% (wt/wt), based on total weight of the dosage form. In certain embodiments, the compositions include active ingredient(s) at 1% to 50% (wt/wt), 5% to 50% (wt/wt), 10% to 50% (wt/wt), 15% to 50% (wt/wt/), 20% to 50% (wt/wt), 25% to 50% (wt/wt), 30% to 50% (wt/wt), 35% to 50% (wt/wt), 40% to 50% (wt/wt), 45% to 50% (wt/wt), 1% to 40% (wt/wt), 1% to 35% (wt/wt), 1% to 30% (wt/wt), 1% to 25% (wt/wt), 1% to 20% (wt/wt), 1% to 15% (wt/wt), 1% to 10% (wt/wt), 1% to 5% (wt/wt), 10% to 40% (wt/wt), 10% to 30% (wt/wt), 10% to 25% (wt/wt), 10% to 20% (wt/wt), 10% to 15% (wt/wt), 15% to 30% (wt/wt), 15% to 25% (wt/wt), 15% to 20% (wt/wt), 1% to 60% (wt/wt), 10% to 60% (wt/wt), 20% to 60% (wt/wt), 30% to 60% (wt/wt), or 40% to 60% (wt/wt), based on total weight of the dosage form.

In certain embodiments, the compositions provide, independently for each active ingredient, a dosage of 0.001 mg to 1000 mg per kg of the subject, preferably 0.01 mg to 100 mg per kg of the subject, more preferably 0.1 mg to 50 mg per kg of the subject, and even more preferably 0.1 mg to 10 mg per kg of the subject.

In certain embodiments, the compositions include, independently for each active ingredient, 0.01 mg to 1000 mg of active ingredient, preferably 0.1 to 500 mg of active ingredient, more preferably 0.5 mg to 100 mg of active ingredient. In a further embodiment, the compositions may include, independently for each active ingredient, 0.03 mg to 1000 mg of active ingredient or 0.06 mg to 500 mg of active ingredient.

In a preferred embodiment, the compositions include a combination of an isoxazoline class parasiticide (e.g., fluralaner, afoxolaner, sarolaner, or lotilaner), a macrocyclic lactone class parasiticide (e.g., milbemycin oxime, moxidectin, doramectin, or selamectin), and a pyrazinoisoquinoline class parasiticide (e.g., praziquantel). In another preferred embodiment, the compositions include a combination of an isoxazoline class parasiticide (e.g., fluralaner, afoxolaner, sarolaner, or lotilaner), a macrocyclic lactone class parasiticide (e.g., milbemycin oxime, moxidectin, or selamectin), a pyrazinoisoquinoline class parasiticide (e.g., praziquantel), and a tetrahydropyrimidine class parasiticide (e.g., pyrantel pamoate, pyrantel tartrate, morantel tartrate, or oxantel embonate).

In yet another preferred embodiment, the compositions include a combination of lotilaner, moxidectin, and praziquantel. In yet another preferred embodiment, the compositions include a combination of lotilaner, milbemycin oxime, and praziquantel. In yet another preferred embodiment, the compositions include a combination of lotilaner, moxidectin, praziquantel, and pyrantel or a salt thereof. In yet another preferred embodiment, the compositions include a combination of lotilaner, milbemycin oxime, praziquantel, and pyrantel or a salt thereof.

In yet another preferred embodiment, the compositions include a combination of 0.5% to 40% (wt/wt) of an isoxazoline (e.g., lotilaner, sarolaner, fluralaner, or afoxolaner), 0.005% to 1% (wt/wt) of a macrocyclic lactone (e.g., moxidectin, milbemycin oxime, doramectin, or selamectin), and 1% to 10% (wt/wt) of a pyrazinoisoquinoline (e.g., praziquantel), based on total weight of the dosage form.

In yet another preferred embodiment, the compositions provide a dosage of 1 mg/kg to 100 mg/kg of an isoxazoline (e.g., lotilaner, sarolaner, fluralaner, or afoxolaner), 0.001 mg/kg to 15 mg/kg of a macrocyclic lactone (e.g., moxidectin, milbemycin oxime, doramectin, or selamectin), and 1 mg/kg to 100 mg/kg of a pyrazinoisoquinoline (e.g., praziquantel).

In yet another preferred embodiment, the compositions include a combination of 0.5% to 40% (wt/wt) of an isoxazoline (e.g., lotilaner, sarolaner, fluralaner, or afoxolaner), 0.005% to 1% (wt/wt) of a macrocyclic lactone (e.g., moxidectin, milbemycin oxime, doramectin, or selamectin), 1% to 10% (wt/wt) of a pyrazinoisoquinoline (e.g., praziquantel), and 5% to 30% (wt/wt) of a tetrahydropyrimidine (e.g., pyrantel pamoate, pyrantel tartrate, morantel tartrate, or oxantel embonate), based on total weight of the dosage form.

In yet another preferred embodiment, the compositions provide a dosage of 1 mg/kg to 100 mg/kg of an isoxazoline (e.g., lotilaner, sarolaner, fluralaner, or afoxolaner), 0.001 mg/kg to 15 mg/kg of a macrocyclic lactone (e.g., moxidectin, milbemycin oxime, doramectin, or selamectin), 1 mg/kg to 100 mg/kg of a pyrazinoisoquinoline (e.g., praziquantel), and 1 mg/kg to 100 mg/kg of a tetrahydropyrimidine (e.g., pyrantel pamoate, pyrantel tartrate, morantel tartrate, or oxantel embonate).

In yet another preferred embodiment, the compositions include a combination of 0.5% to 40% (wt/wt) an isoxazoline (e.g., lotilaner, sarolaner, fluralaner, or afoxolaner), 0.005% to 1% (wt/wt) moxidectin, and 1% to 10% (wt/wt) praziquantel, based on total weight of the dosage form.

In yet another preferred embodiment, the compositions provide a dosage of 1 mg/kg to 100 mg/kg of an isoxazoline (e.g., lotilaner, sarolaner, fluralaner, or afoxolaner), 0.001 mg/kg to 5 mg/kg of moxidectin, and 1 mg/kg to 30 mg/kg of praziquantel.

In yet another preferred embodiment, the compositions include a combination of 0.5% to 40% (wt/wt) lotilaner, 0.005% to 1% (wt/wt) moxidectin, and 1% to 10% (wt/wt) praziquantel, based on total weight of the dosage form. In yet another preferred embodiment, the compositions include a combination of 30-40% (wt/wt) lotilaner, 0.025% to 0.045% (wt/wt) moxidectin, and 8-10% (wt/wt) praziquantel, based on total weight of the dosage form.

In yet another preferred embodiment, the compositions provide a dosage of 1 mg/kg to 100 mg/kg of lotilaner, 0.001 mg/kg to 5 mg/kg of moxidectin, and 1 mg/kg to 30 mg/kg of praziquantel. In yet another preferred embodiment, the compositions provide a dosage of 15 mg/kg to 45 mg/kg of lotilaner, 0.025 mg/kg to 0.50 mg/kg of moxidectin, and 1 mg/kg to 15 mg/kg of praziquantel. In yet another preferred embodiment, the compositions provide a dosage of 20 mg/kg to 40 mg/kg of lotilaner, 0.05 mg/kg to 0.10 mg/kg of moxidectin, and 5 mg/kg to 10 mg/kg of praziquantel. In yet another preferred embodiment, the compositions provide a dosage of 20 mg/kg to 40 mg/kg of lotilaner, 0.02 mg/kg to 0.04 mg/kg of moxidectin, and 5 mg/kg to 10 mg/kg of praziquantel.

In yet another preferred embodiment, the compositions include 20-1,000 mg of lotilaner, 0.001-200 mg of moxidectin, and 5-250 mg of praziquantel. In yet another preferred embodiment, the compositions include 55-57 mg of lotilaner, 0.1-0.2 mg of moxidectin, and 13-15 mg of praziquantel. In yet another preferred embodiment, the compositions include 111-113 mg of lotilaner, 0.2-0.3 mg of moxidectin, and 28-30 mg of praziquantel. In yet another preferred embodiment, the compositions include 224-226 mg of lotilaner, 0.5-0.6 mg of moxidectin, and 56-58 mg of praziquantel. In yet another preferred embodiment, the compositions include 449-451 mg of lotilaner, 1.1-1.2 mg of moxidectin, and 113-115 mg of praziquantel. In yet another preferred embodiment, the compositions include 899-901 mg of lotilaner, 2.2-2.3 mg of moxidectin, and 227-229 mg of praziquantel.

In yet another preferred embodiment, the compositions include a combination of 0.5% to 40% (wt/wt) of an isoxazoline (e.g., lotilaner, sarolaner, fluralaner, or afoxolaner), 0.005% to 1% (wt/wt) moxidectin, 1% to 10% (wt/wt) praziquantel, and 5% to 30% (wt/wt) pyrantel pamoate, based on total weight of the dosage form.

In yet another preferred embodiment, the compositions include a combination of 20-40% (wt/wt), preferably 20-30% (wt/wt) lotilaner, 0.02-0.045%, preferably 0.02-0.03 (wt/wt) moxidectin, and 5-10% (wt/wt), preferably 5-7% (wt/wt) praziquantel, and 5% to 30% (wt/wt), preferably 10% to 20% (wt/wt) pyrantel pamoate based on total weight of the dosage form. In yet another preferred embodiment, the compositions provide a dosage of 1 mg/kg to 100 mg/kg of an isoxazoline (e.g., lotilaner, sarolaner, fluralaner, or afoxolaner), 0.001 mg/kg to 5 mg/kg of moxi dectin, 1 mg/kg to 30 mg/kg of praziquantel, and 1 mg/kg to 30 mg/kg of pyrantel pamoate.

In yet another preferred embodiment, the compositions provide a combination of 0.5% to 40% (wt/wt) lotilaner, 0.005% to 1% (wt/wt) moxidectin, 1% to 10% (wt/wt) praziquantel, and 5% to 30% (wt/wt) pyrantel pamoate, based on total weight of the dosage form.

In yet another preferred embodiment, the compositions provide a dosage of 1 mg/kg to 100 mg/kg of lotilaner, 0.001 mg/kg to 5 mg/kg of moxidectin, 1 mg/kg to 30 mg/kg of praziquantel, and 1 mg/kg to 30 mg/kg of pyrantel pamoate. In yet another preferred embodiment, the compositions provide a dosage of 15 mg/kg to 45 mg/kg of lotilaner, 0.025 mg/kg to 0.50 mg/kg of moxidectin, 1 mg/kg to 15 mg/kg of praziquantel, and 1 mg/kg to 15 mg/kg of pyrantel pamoate. In yet another preferred embodiment, the compositions provide a dosage of 20 mg/kg to 40 mg/kg of lotilaner, 0.05 mg/kg to 0.10 mg/kg of moxidectin, 5 mg/kg to 10 mg/kg of praziquantel, and 5 mg/kg to 10 mg/kg of pyrantel pamoate. In yet another preferred embodiment, the compositions provide a dosage of 20 mg/kg to 40 mg/kg of lotilaner, 0.02 mg/kg to 0.04 mg/kg of moxidectin, 5 mg/kg to 10 mg/kg of praziquantel, and 5 mg/kg to 10 mg/kg of pyrantel pamoate.

In yet another preferred embodiment, the compositions include 20-1,000 mg of lotilaner, 0.001-200 mg of moxidectin, 5-250 mg of praziquantel, and 5-250 mg of pyrantel pamoate.

### Protected Active Ingredients

In certain embodiments, one or more actives of the compositions may be incorporated into the dosage form as a coated granulate. Actives may be incorporated in the dosage forms as coated granulates to, for example, taste-mask unpalatable ingredients or shield a labile active ingredient from other actives or excipients in the dosage form matrix.

The coated granulates can be coated with, for example, a physiologically acceptable polymer matrix. The physiologically acceptable polymer matrix may include one or more polymers and optionally one or more plasticizers.

Suitable classes of polymer include, but are not limited to, shellac, a polymer on a cellulose, acrylic acid or methacrylic acid, maleic acid anhydride, polyvinyl pyrrolidone or polyvinyl alcohol basis. Other polymers may also be considered, e.g. polymers on a cellulose basis, e.g. produced from cellulose acetate phthalate or cellulose acetate-N,N-di-n-butylhydroxypropylether. The starting materials for polymers on an acrylic acid or methacrylic acid basis may be methacrylate/methacrylic acid copolymer, 2-methyl-5-vinyl-pyridine/methacrylate/methacrylic acid copolymer, methyl methacrylate/methacrylic acid copolymer, methyl methacrylate/methacrylic acid copolymer, methyl methacrylate/maleic acid anhydride copolymer, or methyl methacrylate/maleic acid anhydride copolymer.

Polymers on an acrylic acid or methacrylic acid basis are preferably used, e.g. polymerization products of acrylic acid and acrylic acid esters with a low content of quaternary ammonium groups, e.g. as commercially available under the names Eudragit® E, L or S from the company Rohm, (now Evonik) Darmstadt, Germany. Eudragit® E is a cationic polymer of dimethylaminoethyl methacrylate and a neutral methacrylic acid ester. Eudragit®L and S are anionic copolymers of methacrylic acid and methacrylic acid methylester. Eudragit®E 100 is a pH-dependent cationic polymer, which dissolves in the gastric juices at an acidic pH value of up to pH 5.0. Above pH 5.0, it is capable of swelling. In powder form, it is known and commercially available as Eudragit® EPO. Eudragit® EPO has the advantage that the process can be carried out in an aqueous medium and optionally without organic solvents.

In a preferred embodiment, the physiologically acceptable polymer matrix comprises a poly(meth)acrylate polymer such as Eudragit® RL 100, Eudragit® RL PO, Eudragit® RL 30D, Eudragit® RL 12.5, Eudragit® RS 100, Eudragit® RS PO, Eudragit® RS 30D, Eudragit® RS 12.5, Eudragit® NE 30D, or Eudragit® NE 40D, all available from Evonik.

In an even more preferred embodiment, the physiologically acceptable polymer matrix comprises Eudragit® RL (which may for example be used as an aqueous dispersion of Eudragit® RL 100 with 30% dry substance: Eudragit® RL 30D) Eudragit® RL is a copolymer of acrylic and methacrylic esters with a low content of quaternary ammonium groups, the molar ratio of ammonium groups to the remaining neutral (meth)acrylic esters being 1:20 in Eudragit® RL. The mean molecular weight is about 32,000. The code designation RL refers to the light retardation properties of the agent.

Suitable plasticizers for inclusion in the physiologically acceptable polymer matrix include, for example, triethyl citrate, polyethylene glycol, dibutyl phthalate, diethylphthalate and triacetin. In certain embodiments, the plasticizer is provided as a 20% aqueous emulsion comprising glycerol monostearate, triethyl citrate, and polysorbate 80 (e.g., PlasACRYL® HTP20; Evonik Corporation). In a preferred embodiment, PlasACRYL® HTP20 is formulated with EUDRAGIT® RL 30 D to provide the physiologically acceptable polymer matrix coating the granulate materials.

Coating as used herein includes a full or partial coating of the material; therefore, according to one embodiment, coated material as described herein may refer to a partially coated agglomerate.

Coating of the active-containing granulate can be affected by spraying a solution or suspension of polymer-containing material onto the granulate. Volatile components (e.g., water or an organic solvent) can be subsequently removed (e.g. by application of heat and/or under vacuum). After the drying process, the coated granulate may be sieved.

Suitable solvents for dissolution of the polymer are, for example, solvents which are relatively readily volatile, e.g. one or more of the following: methanol, ethanol, isopropanol, butanol, benzyl alcohol, ethylene glycol, propylene glycol, phenol, acetone, acetic acid, acetic acid anhydride, nitromethane, ethylene diamine, acetic acid cellosolve, e.g. an acetone—ethanol mixture, e.g. in a ratio of 1:1. Aqueous suspensions or solutions may be used, for example coating may be carried out with Eudragit® EPO or Eudragit® RL 30D from an aqueous dispersion. According to this process, safety aspects, environmental protection and economical advantages are optimally combined.

### Excipients

The compositions optionally include one or more acceptable excipients. The term “acceptable excipient” refers to those typically used in preparing veterinary and pharmaceutical compositions and preferably are pure and non-toxic in the amounts used. They generally are a solid, semisolid, or liquid material, which in the aggregate can serve as a vehicle or medium for the active ingredient. Some examples of acceptable excipients are found in Remington's Pharmaceutical Sciences and the Handbook of Pharmaceutical Excipients and include diluents, vehicles, carriers, ointment bases, binders, disintegrants, lubricants, glidants, sweetening agents, flavoring agents, gel bases, sustained release matrices, stabilizing agents, preservatives, solvents, suspending agents, buffers, emulsifiers, dyes, propellants, coating agents, and others.

The present compositions may comprise one or more flavouring agents to improve the palatability. In particular, the compositions may comprise a meat flavouring. Exemplary flavorants include, but are not limited to, Flavorpal® flavourings, Desiccated Pork Liver Powder™, Provesta® 356, Provesta® 400, PC-0125, Symtripal® flavourings, for example Symtripal® chicken cooked dry flavor, Symtripal® meat chopped type dry flavor, Symtripal® liver type dry flavor and Symtripal® chicken skin and meat dry flavor. As an additional example, Diana Pet Food's “D'TECH 8P0910748 VEGGIE” may be mentioned which is marketed as a “palatability enhancer”. Meat or beef flavorings used in exemplary embodiments may be naturally derived or artificially formulated to have a meat or beef flavor. Natural flavorings are often made up of dried and pulverized or powdered meat which may be obtained from domesticated meat animals including cattle such as cows or bulls, pigs, deer, sheep, goats, poultry which may include turkey, chicken, duck and the like. Non-animal, often plant derived flavorings, include soy, peanuts, fruits, sweeteners, honey, sugar, maple syrup and fructose, parsley, celery, peppermint, spearmint, garlic, and the like. In exemplary embodiments, flavorants may optionally be included in the compositions, for example, in concentrations of 0.5% to 40% (wt/wt), based on total weight of the dosage form. In a preferred embodiment, the flavouring is Symtripal® meat type dry flavor or Symtripal® liver type dry flavor. In another preferred embodiment the flavoring is Symtripal® liver type dry flavor in combination with D'TECH 8P0910748 VEGGIE. In a preferred embodiment, the composition comprises 0.5 to 20% (wt/wt), preferably 5 to 15% (wt/wt) of flavoring, which may be one flavoring or a combination of at least two flavorings.

The present compositions may comprise a surfactant. A surfactant (also known as a surfaceactive agent) is a component which lowers surface tension at the interface between two liquids or between a liquid and solid. Suitable surfactants can be found in REMINGTON: THE SCIENCE AND PRACTICE OF PHARMACY (20^{th} ed., Mack Publishing Co., 2001). Exemplary surfactants include, but are not limited to, glyceryl monooleate, polyoxyethylene sorbitan fatty acid esters, sorbitan esters including sorbitan monooleate (Span® 20), polyvinyl alcohol, polysorbates including polysorbate 20 and polysorbate 80, d-a-tocopheryl polyethylene glycol 1000 succinate (TPGS), Vitamin E TPGS (D-a-Tocopheryl polyethylene glycol 1000 succinate), sodium lauryl sulfate (SLS), co-polymers of ethylene oxide and propylene oxide (e.g. poloxamers such as Poloxamer 124, 188, 338, and 407, and LUTROL® F87 and the like), polyethylene glycol castor oil derivatives including polyoxyl 35 castor oil (Cremophor® EL), polyoxyl 40 hydrogenated castor oil (Cremophor® RH 40), polyoxyl 60 hydrogenated castor oil (Cremophor® RH60); propylene glycol monolaurate (LAUROGLYCOL®); glyceride esters including glycerol capryl ate/caprate (CAPMUL® MCM), polyglycolized glycerides (GELUCIRE®, such as Gelucire® 44/14), PEG 300 caprylic/capric glycerides (Softigen® 767), PEG 400 caprylic/capric glycerides (Labrasol®), PEG 300 oleic glycerides (Labrafil® M-1944CS), PEG 300 linoleic glycerides (Labrafil® M-2125CS); polyethylene glycol stearates and polyethylene glycol hydroxy stearates including polyoxyl 8 stearate (PEG 400 monostearate), polyoxyl 40 stearate (PEG 1750 monostearate), PEG3350, and the like. Polyethylene glycol stearates (synonyms include macrogol stearates, polyoxyl stearates, polyoxyethylene stearates, ethoxylated stearates; CAS No. 9004-99-3, 9005-08-7) are mixtures of mono- and distearate esters of mixed polyoxyethylene polymers. Polyethylene glycol hydroxy stearate is a mixture of mono- and diesters of hydroxystearic acid with polyethylene glycols. One polyethylene glycol hydroxystearate that may be used in the compositions is polyethylene glycol 12-hydroxy stearate. The compositions may include the surfactant polyethylene glycol 15 12-hydroxystearate (Solutol® HS 15 from BASF), a mixture of mono- and diesters of 12-hydroxy stearic acid with 15 moles of ethylene oxide. The compositions may include polyoxyl 35 castor oil (Cremophor® EL) as a surfactant. The compositions may include polyoxyl 40 hydrogenated castor oil (Cremophor® RH 40) or polyoxyl 60 hydrogenated castor oil (Cremophor® RH60) as surfactants. In exemplary embodiments, surfactants may optionally be included in the compositions, for example, in concentrations of about 0.1% to about 10% (wt/wt), or 0.005% to about 5% (wt/wt), or 0.01% to about 1% (wt/wt)based on total weight of the dosage form. In a further embodiment, the composition comprises 0.5 to 5% (w/w) or 0.01% to about 5% (wt/wt), preferably 0.01% to about 1% (wt/wt) sodium lauryl sulfate.

The present compositions may comprise a solvent. Exemplary solvents include, but are not limited to, glycerol anhydrous (CAS 56-81-5), various grades of liquid polyethylene glycol (PEG) including PEG 200, PEG 300, PEG 400 and PEG 540; propylene carbonate; propylene glycol; triglycerides including, but not limited to caprylic/capric triglyceride, caprylic/capric/linoleic triglyceride (e.g. MIGLYOL® 810 and 812), caprylic/capric/succinic triglyceride, propylene glycol di capryl ate/di caprate, and the like; water, sorbitol solution, glycerol caprylate/caprate and polyglycolized glycerides (GELUCIRE®), or a combination thereof. Further exemplary solvents include medium chain triglycerides (MCTs) and vegetable oils termed long-chain triglycerides (LCTs). Triglycerides of medium-chain length fatty acids, known as medium-chain triglycerides or MCTs, can be synthesized by esterifying glycerol with fatty acids of carbon chain lengths of C8 or CIO. MCTs are usually commercially-available as a mixture of glycerol esters of C8 (octanoic acid or caprylic acid) and CIO (decanoic acid or capric acid) fatty acids, with small amounts (<1% of each) of glycerol esters of C6 (hexanoic acid or caproic acid) and C12 (dodecanoic acid or lauric acid) fatty acids. In exemplary embodiments, solvents may optionally be included in the compositions, for example, in concentrations of about 1 to about 50% (wt/wt), based on total weight of the dosage form. According to a further embodiment the present compositions are essentially free of solvents (where “essentially free” means that no solvents are added as an ingredient per se but the composition may contain traces of solvents which may be present in the various components or may remain from any production steps).

The present compositions may comprise a filler. Exemplary fillers include, but are not limited to, corn starch, pre-gelatinized corn starch, soy protein fines, corn cob, corn gluten meal, dibasic calcium phosphate hydrous or anhydrous, microcrystalline cellulose (MCC), and lactose hydrous or anhydrous. In exemplary embodiments, fillers may optionally be included in the compositions, for example, in concentrations of about 5% to about 80% (wt/wt).

The present compositions may comprise a binder. A binder is a component which increases cohesiveness. Suitable binders can be found in REMINGTON: THE SCIENCE AND PRACTICE OF PHARMACY (20^{th} ed., Mack Publishing Co., 2001). Exemplary binders include, but are not limited to, polyvinylpyrrolidone (e.g. Povidone and Plasdone® K-29/32) or K30), cross-linked polyvinylpyrrolidone (Crospovidone), polyethylene glycols of various grades including PEG 3350, PEG 4000, PEG 6000, PEG 8000 and even PEG 20,000, and the like; copolymers of vinylpyrrolidone and vinyl acetate (e.g. Copovidone) such as the product sold by BASF by the tradename Kollidon® VA 64 and the like; starch such as potato starch, tapioca starch or corn starch; molasses, corn syrup, honey, maple syrup and sugars of various types; hydroxypropyl cellulose (HPC), hydroxypropyl methylcellulose (HPMC), and alginate. In exemplary embodiments, binders may optionally be included in the compositions, for example, in concentrations of about 1% to about 30% (wt/wt) or 0.1% to about 10% (wt/wt), preferably 0.1% to about 5% (wt/wt), more preferably 0.2% to about 3% (wt/wt). In a preferred embodiment one of the above-mentioned polyvinylpyrrolidone binders (e.g. PVP K30) is present.

The present compositions may comprise an antioxidant. “Antioxidants” are components which serve as preservatives to increase the stability of active ingredients which are not stable if exposed to oxygen. Suitable antioxidants can be found in REMINGTON: THE SCIENCE AND PRACTICE OF PHARMACY (20^{th} ed., Mack Publishing Co., 2001). Suitable antioxidants include, but are not limited to, Tenox® 2; Tenox® PG; Tenox® s-1; BHA (2-t-butyl-4-methoxyphenol); BHT (2,6-di-t-butyl-4-methylphenol); sodium metabisulfite reducing agents; antioxidant synergists such as tocopherols (alpha, beta, or delta-tocopherol, tocopherol esters, alpha-tocopherol acetate), alkyl gallates, butylated hydroxyanisole, butylated hydroxytoluene, citric acid anhydrous and hydrous, edetic acid and its salts, lecithin, tartaric acid, ascorbic acid, ascorbyl palmitate, fumaric acid, malic acid, sodium ascorbate, sodium metabi sulfate, n-propyl gallate, monothioglycerol, resveratrol, quercetin, benzoic acid, dimethyl thiourea (DMTU), hesperetin, tetrahydrocurcumin, tetrahydrodemethoxycurcumin, and Trolox (N-acetylcysteine, 6-hydroxy-2,5,7,8-tetramethylchroman-2-carboxylic acid). In a preferred embodiment, the antioxidant is BHA. In exemplary embodiments, antioxidants may optionally be included in the compositions, for example, in concentrations of about 0.01 to about 2.0% (wt/wt), preferably 0.01 to about 0.1% (wt/wt), based upon total weight of the dosage form. In a further embodiment, the composition comprises 0.01 to 0.2% (wt/wt) BHA. In a further embodiment, the composition comprises 0.01 to 0.2% (wt/wt) BHT, preferably 0.01 to 0.1% (wt/wt) BHT. The present compositions may comprise a lubricant. Exemplary lubricants include, but are not limited to, polyethylene glycols of various molecular weight ranges including PEG 3350 (Dow Chemical) and PEG 4000, corn oil, mineral oil, hydrogenated vegetable oils (STEROTEX or LUBRITAB), peanut oil and/or castor oil. In certain embodiments, the lubricant is a neutral oil comprising a medium chain triglyceride (MCT) or propylene glycol fatty acid esters including caprylic/capric triglycerides. Non-limiting examples of neutral oils are known by the trademark MIGLYOL® including MIGLYOL® 810, MIGLYOL® 812, MIGLYOL® 818, MIGLYOL® 829 and MIGLYOL® 840. Further lubricants include vegetable oils termed long-chain triglycerides (LCT), magnesium stearate (MgSt), stearic acid, and sodium stearyl fumarate. In exemplary embodiments, lubricants may optionally be included in the compositions, for example, in concentrations of about 1% to about 50% (wt/wt), or 0.1% to about 10% (wt/wt), preferably 1% to about 5% (wt/wt), based on total weight of the dosage form.

The present compositions may comprise a glidant. Exemplary flow aids or glidants include, but are not limited to, silicon dioxide (e.g. CARBOSILor Aerosil) or silica gel (SYLOID), talc, starch, calcium, stearate, magnesium stearate, and aluminum magnesium silicate (NEUSILIN).

In exemplary embodiments, glidants may optionally be included in the compositions, for example, in concentrations of about 0.01 to about 25%, based upon weight of total composition. The present compositions may comprise one or more palatability enhancers.

The present compositions may comprise a sweetener to further improve the palatability. Any natural sugar may be used including confectioners' sugar, maltitol, xylitol, sorbitol, mannitol, lactose, dextrose, saccharose, glucose or fructose, or any mixture thereof. Artificial sweeteners may also be used including saccharins, aspartame, acesulfame-k and Rebaten®.

The present compositions may comprise a coloring agent. The coloring agent may be selected from azo dyes, organic or inorganic pigments or coloring agents of natural origin. The composition may comprise 0.05 to 0.25% (w/w) of a coloring agent, based on total weight of the dosage form.

The present compositions may comprise a disintegrant. Exemplary disintegrants include, but are not limited to, cellulose, carboxymethyl cellulose calcium, carboxymethyl cellulose sodium, polacrilin potassium, starch, hydroxypropyl starch, corn starch, bentonite, veegum, pregelatinized starch, modified starch, lactose monohydrate, croscarmellose sodium, microcrystalline cellulose, hydroxypropyl cellulose, glycine, crospovidone, magnesium aluminum silicate, sodium starch glycolate, guar gum, colloidal silicon dioxide, polyvinylpyrrolidone (Povidone), alginic acid, sodium alginate, calcium alginate, methylcellulose, and chitosan. In exemplary embodiments, disintegrants may optionally be included in the compositions, for example, in concentrations of 0.1% (wt/wt) to about 10% (wt/wt), based on total weight of the dosage form.

### 3. PROCESSES TO MAKE COMPOSITIONS

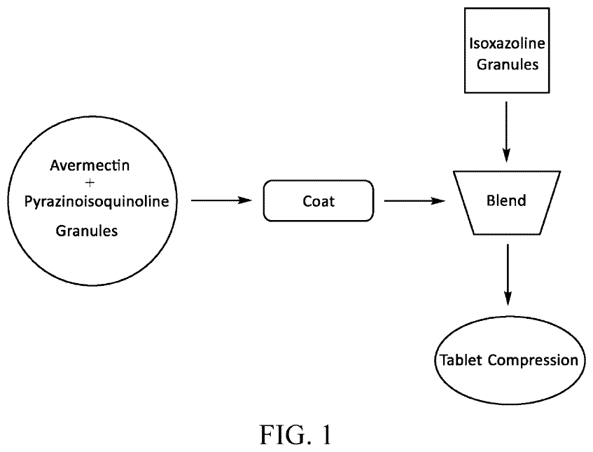

In another aspect, processes are provided for making the disclosed compositions. In one exemplary embodiment, as shown in FIG. 1, the compositions can be prepared by providing a first granulate comprising pyrazinoisoquinoline and avermectin actives; optionally coating the first granulate with a physiologically acceptable polymer matrix (e.g., a polymethacrylate-based copolymer); providing a second granulate comprising an isoxazoline class parasiticide; combining the first and second granulates and at least one pharmaceutically acceptable excipient to provide a final blend; and compressing the ready-to-press mixture into a tablet.

According to a further exemplary embodiment, compositions according to the present invention can be prepared by providing a coated first granulate comprising pyrazinoisoquinoline (e.g. praziquantel) and avermectin actives (e.g. moxidectin) as described above, providing a second granulate comprising an isoxazoline class parasiticide (e.g. lotilaner) and pyrantel or a salt thereof (e.g. pyrantel pamoate), combining the first and second granulates and at least one pharmaceutically acceptable excipient to provide a final blend; and compressing the ready-to-press mixture into a tablet.

### 4. PROPERTIES

The disclosed compositions can be characterized by one or more properties. For example, the compositions can be characterized by disintegration, friability, hardness, and palatability. “Ph. Eur.” means the European Pharmacopeia. If the version is not specified this abbreviation refers to the European Pharmacopeia 10.0.

### Disintegration

Disintegration of the disclosed compositions can be assessed according to disintegration method 2.9.1 (Test A for smaller tablets and B for larger tablets) of the European Pharmacopoeia 6.0. Alternatively, disintegration can be assessed according to USP <701> DISINTEGRATION. Preferred compositions of the present disclosure disintegrate in less than 15 minutes in water at 37° C., as determined by the disintegration method 2.9.1 (Test A and B) of the European Pharmacopoeia 8.0. More preferred compositions of the present disclosure disintegrate in less than 10 minutes in water at 37° C., as determined by the disintegration method 2.9.1 (Test A and B) of the European Pharmacopoeia 8.0. Yet more preferred compositions of the present disclosure disintegrate in less than 5 minutes in water at 37° C., as determined by the disintegration method 2.9.1 (Test A and B) of the European Pharmacopoeia. Yet even more preferred compositions of the present disclosure disintegrate in less than 2 minutes in water at 37° C., as determined by the disintegration method 2.9.1 (Test A and B) of the European Pharmacopoeia.

The compositions may have a disintegration time of less than or equal to 15 minutes, less than or equal to 10 minutes, less than or equal to 5 minutes, less than or equal to 4 minutes, less than or equal to 3 minutes, less than or equal to 2 minutes, or less than or equal to 1 minute measured in water at 37° C., as determined by the disintegration method 2.9.1 (Test A and B) of the European Pharmacopoeia 6.0.

### Friability

Friability of the disclosed compositions can be assessed according to European Phamacopoeia 10.0, Ph. Eur.2.9.7 or preferably USP <1216> TABLET FRIABILITY (.

The compositions can have a friability of 1% or less, 0.5% or less, 0.1% or less, or 0.05% or less, as measured according to USP <1216>. The compositions can have a friability of 1.0%, 0.5%, 0.1%, 0.09%, 0.08%, 0.07%, 0.06%, 0.05%, 0.04%, 0.03%, 0.02%, or 0.01%, as measured according to USP <1216>.

### Hardness

Hardness of the disclosed compositions can be assessed according to Ph. Eur. 2.9.8. Resistance to crushing or preferably USP <1217> TABLET BREAKING FORCE.

The compositions can have a hardness of 6-9 kiloPascal (kP), 6-8 kP, or 6-7 kP, as measured according to USP <1217>. The compositions can have a hardness of 6 kP, 7 kP, 8 kP, or 9 kP, as measured according to USP <1217>. According to a further embodiment the present compositions can have a hardness of 1-50 kiloPascal (kP), 1-40 kP, or 1-35 kP, as measured according to USP <1217>.

### Palatability

Palatability of the disclosed compositions can be assessed according to the Guideline on the demonstration of palatability of veterinary medicinal products, effective February 2015 [EMA/CVMP/EWP/206024/2011 (2014)], Committee for Medicinal Products for Veterinary Use (CVMP).

The compositions can have a palatability of greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 50%, or greater than or equal to 55% as measured according to the Guideline on the demonstration of palatability of veterinary medicinal products, effective February 2015 [EMA/CVMP/EWP/206024/2011 (2014)], Committee for Medicinal Products for Veterinary Use (CVMP). The compositions can have a palatability of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, as measured according to the Guideline on the demonstration of palatability of veterinary medicinal products, effective February 2015 [EMA/CVMP/EWP/206024/2011 (2014)], Committee for Medicinal Products for Veterinary Use (CVMP).

### 5. METHODS OF USE

The compositions of the present disclosure can be used to treat and/or control ectoparasite infections or infestations in an animal. The compositions can be used as an ectoparasiticide, in particular, as an acaricide and insecticide. They may, in particular, be used in the fields of veterinary medicine, livestock husbandry and the maintenance of public health: against acarids, insects, and copepods which are parasitic upon vertebrates, particularly warm-blooded vertebrates, including companion animals, livestock, and fowl and cold-blooded vertebrates like fish. Non-limiting examples of ectoparasites include: ticks (e.g., Ixodes spp., (e.g., L. ricinus, L. hexagonus), Rhipicephalus spp. (e.g., R. sanguineus), Boophilus spp., Amblyomma spp. (e.g., A. americanum, A. maculatum, A. triste, A. parvum, A. cajennense, A. ovale, A. oblongoguttatum, A. aureolatum, A. cajennense), Hyalomma spp., Haemaphysalis spp., Dermacentor spp. (e.g., D. variabilis, D. andersoni, D. marginatus), Ornithodorus spp., and the like); mites (e.g., Dermanyssus spp., Sarcoptes spp. (e.g., S. scabiei), Psoroptes spp. (e.g., P. bovis), Otodectes spp., Chorioptes spp., Demodex spp., (e.g., D. folliculorum, D. canis, and D. brevis) and the like); chewing and sucking lice (e.g., Damalinia spp., Linognathus spp., Cheyletiella spp., Haematopinus spp., Solenoptes spp., Trichodectes spp., Felicola spp., and the like); fleas (e.g., Siphonaptera spp., Ctenocephalides spp., and the like); biting flies, midges, and mosquitos (e.g., Tabanidae spp., Haematobia spp., Musca spp., Stomoxys spp., Dematobia spp., Cochliomyi a spp., Simuliidae spp., Ceratopogonidae spp., Psychodidae spp., Aedes spp., Culex spp., Anophl es spp., Lucilia spp., Phlebotomus spp., Lutzomyia spp., and the like); bed bugs (e.g., insects within the genus Cimex and family Cimicidae); and grubs (e.g., Hypoderma bovis, H. lineatum); and copepods (e.g., sea lice within the Order Siphonostomatoida, including genera Lepeophtheirus and Caligus).

The compositions of the present disclosure can be used to treat and/or control endoparasites, for example, helminths (e.g., trematodes, cestodes, and nematodes) including heartworm, roundworm, hookworm, whipworm, fluke, and tapeworm. The gastrointestinal roundworms include, for example, Ostertagia ostertagi (including inhibited larvae), O. lyrata, Haemonchus placei, H. similis, H. contortus, Toxocara canis, T. leonina, T. cati, Trichostrongylus axei, T. colubriformis, T. longispicularis, Cooperia oncophora, C. pectinata, C. punctata, C. surnabada (syn. mcmasteri), C. spatula, Ascaris suum, Hyostrongylus rubidus, Bunostomum phlebotomum, Capillaria bovis, B. trigonocephalum, Strongyloides papillosus, S. ransomi, Oesophagostomum radiatum, O. dentatum, O. columbianum, O. quadrispinulatum, Trichuris spp., and the like. Other parasites include: hookworms (e.g., Ancylostoma caninum, A. tubaeforme, A. braziliense, Uncinaria stenocephala); lungworms (e.g., Dictyocaulus viviparus and Metastrongylus spp); eyeworms (e.g., Thelazia spp.); parasitic stage grubs (e.g., Hypoderma bovis, H. lineatum, Dermatobia hominis; kidneyworms (e.g., Stephanurus dentatus); screw worm (e.g., Cochliomyia hominivorax (larvae); filarial nematodes of the super-family Filarioidea and the Onchocercidae Family. Non-limiting examples of filarial nematodes within the Onchocercidae Family include the genus Brugia spp. (i.e., B. malayi, B. pahangi, B. timori, and the like), Wuchereria spp. (i.e., W. bancrofti, and the like), Dirofdaria spp. (D. immitis, D. repens, D. ursi, D. tenuis, D. spectans, D. lutrae, and the like), Dipetalonema spp. (i.e., D reconditum, D. repens, and the like), Onchocerca spp. (i.e., O. gibsoni, O. gutturosa, O. volvulus, and the like), Elaeophora spp. (E. bohmi, E. elaphi, E. poeli, E. sagitta, E. schneideri, and the like), Mansonella spp. (i.e., M. ozzardi, M. perstans, and the like), and Loa spp. (i.e., L. loa).

The compositions of the present disclosure are useful for the treatment and/or control, in particular helminths, in which the endoparasitic nematodes and trematodes may be the cause of serious diseases of mammals and poultry. Typical nematodes of this indication are: Filariidae, Setariidae, Haemonchus, Trichostrongylus, Ostertagia, Nematodirus, Cooperia, Ascaris, Bunostonum, Oesophagostonum, Charbertia, Trichuris, Strongylus, Trichonema, Dictyocaulus, Capillaria, Heterakis, Toxocara, Ascaridia, Oxyuris ncylostoma, Uncinaria, Toxascaris and Parascaris. The trematodes include, in particular, the family of Fasciolideae, especially Fasciola hepatica.

Certain parasites of the species Nematodirus, Cooperia and Oesophagostonum infest the intestinal tract of the host animal, while others of the species Haemonchus and Ostertagia are parasitic in the stomach and those of the species Dictyocaulus are parasitic in the lung tissue. Parasites of the families and may be found in the internal cell tissue and in the organs, e.g. the heart, the blood vessels, the lymph vessels and the subcutaneous tissue. A particularly notable parasite is the heartworm of the dog, Dirofdaria iminitis.

The parasites which may be treated and/or controlled by the compositions of the present disclosure include those from the class of Cestoda (tapeworms), e.g. the families Mesocestoidae, especially of the genus Mesocestoides, in particular M. lineatus; Dipylidiidae, especially Dipylidium caninum, Joyeuxiella spp., in particular Joyeuxiella pasquali, and Diplopylidium spp., and Taeniidae, especially Taenia pisformis. Taenia cervi. Taenia ovis, Taeneia hydaligena. Taenia multiceps, Taenia laeniaeformis. Taenia serialis, and Echinococcus spp., most particularly Taneia hydaligena. Taenia ovis, Taenia multiceps, Taenia serialis; Echinococcus granulosus and Echinococcus multilocularis.

Furthermore, the compositions of the present disclosure are suitable for the treatment and/or control of human pathogenic parasites. Of these, typical representatives that appear in the digestive tract are those of the genus Ancylostoma, Necator, Ascaris, Strongyloides, Trichinella, Capillaria, Trichuris and Enterobius. The compositions of the present disclosure are also suitable against parasites of the genus Wuchereria, Brugia, Onchocerca and Loa from the family of Dracunculus and parasites of the genus Strongyloides and Trichinella, which infect the gastrointestinal tract in particular.

A particular parasite to be treated and/or controlled by the compositions of the disclosure is the heartworm (Dirofilaria immitis). Particular subjects for such treatment are dogs and cats.

In certain embodiments, the composition of the present disclosure is suitable for the treatment and/or control of against tick infestations (Ixodes hexagonus, Ixodes ricinus and Rhipicephalus sanguineus, Dermacentor reticulatus), for the treatment and/or control of flea infestations (Ctenocephalides felis and Ctenocephalides canis),

In certain embodiments, the composition of the present disclosure is suitable for the treatment and/or control of adult fleas and prevention of new flea infestations (Ctenocephalidesfelis): the treatment and/or control of tick infestations [Dermacentor variabilis (American dog tick), Ixodes scapularis (black-legged tick), Rhipicephalus sanguineus (brown dog tick), Amblyomma americanum (lone star tick), Dermacentor reticulatus, Ixodes ricinus and Ixodes hexagonus]; the treatment and/or control nematodes: heartworm disease (Dirofilaria immitis), angiostrongylosis by reducing the level of infection with immature adult (L5) Angiostrongylus vasorum, adult hookworm (Ancylostoma caninum), adult roundworm (Toxocara canis Toxascaris leonina) and adult whipworm (Trichuris vulpis) infections, and cestodes (Dipylidium caninum, Taenia pisiformis. Echinococcus multilocularis, and Echinococcus granulosus) infections in dogs 8 weeks of age and older and 4.4 pounds (2 kg) of body weight or greater.

The compositions of the disclosure may be combined with one or more other active compounds or therapies for the treatment of one or more disorders, diseases or conditions, including the treatment of parasites. The compositions of the present disclosure may be administered simultaneously, sequentially or separately in combination with one or more compounds or therapies for treating parasites or other disorders.

For reasons of completeness, various aspects of the disclosure are set out in the following numbered clauses.

1. A palatable veterinary dosage form comprising
an isoxazoline class parasiticide (e.g., lotilaner, sarolaner, afoxolaner, fluralaner); moxidectin; and
praziquantel,
wherein the praziquantel, and optionally moxidectin, are confined to discrete areas within the dosage form.

2. The palatable veterinary dosage form of clause 1, further comprising pyrantel or a salt thereof.

3. The palatable veterinary dosage form of clause 1 or clause 2, wherein the moxidectin and praziquantel are comprised within physiologically acceptable polymer matrix-coated granules (e.g., polymethacrylate-based copolymer-coated granules).

4. The palatable veterinary dosage form of any one of clauses 1-3, wherein the palatable veterinary dosage form is a hard chewable tablet.

5. The palatable veterinary dosage form of any one of clauses 1-4, wherein the palatable veterinary dosage form has a hardness of 6-9 kiloPascal (kP), 6-8 kP, or 6-7 kP, as measured according to USP <1217>.

6. The palatable veterinary dosage form of any one of clauses 1-5, wherein the palatable veterinary dosage form disintegrates in less than or equal to 15 minutes, less than or equal to 10 minutes, less than or equal to 5 minutes, less than or equal to 4 minutes, less than or equal to 3 minutes, or less than or equal to 2 minutes in water at 37° C., as determined by the disintegration method 2.9.1 (Test B) of the European Pharmacopoeia 8.0.

7. The palatable veterinary dosage form of any one of clauses 1-6, wherein the palatable veterinary dosage form has a friability of 1% or less, 0.5% or less, 0.1% or less, or 0.05% or less, as measured according to USP <1216>.

8. The palatable veterinary dosage form of any one of clauses 1-7, wherein the palatable veterinary dosage form has a palatability greater than or equal to 60%, greater than or equal to 65%, greater than or equal to 70%, greater than or equal to 75%, greater than or equal to 80%, greater than or equal to 85%, greater than or equal to 90%, or greater than or equal to 95%, as measured according to the Guideline on the demonstration of palatability of veterinary medicinal products, effective February 2015 [EMA/CVMP/EWP/206024/2011 (2014)], Committee for Medicinal Products for Veterinary Use (CVMP).

9. The palatable veterinary dosage form of any one of clauses 1-8, comprising 30-40% (wt/wt) lotilaner, 0.025% to 0.045% (wt/wt) moxidectin, and 8-10% (wt/wt) praziquantel, based on total weight of the dosage form.

10. The palatable veterinary dosage form of any one of clauses 1-8, further comprising at least one of a diluent (e.g, microcrystalline cellulose); an antioxidant (e.g., butylated hydroxytoluene); a disintegrant (e.g., croscarmellose sodium); a wetting agent (e.g., sodium lauryl sulfate); a binder (e.g., povidone 1K30); a taste-masking polymer (e.g., Eudragit RL 30D); a plasticizer (e.g., Plasacryl HTP20); a flavoring agent (e.g., Dtech 8P0910748 Veggie, Symtripal Liver Type Dry Flavour); a glidant (e.g., colloidal silicon dioxide); or a lubricant (e.g., magnesium stearate).

11. The palatable veterinary dosage form of clause 10, comprising microcrystalline cellulose; lactose monohydrate; butylated hydroxytoluene; croscarmellose sodium; sodium lauryl sulfate; povidone K30; Eudragit RL 30D; Plasacryl HTP20; a flavorant (e.g., Dtech 8P0910748 Veggie, or Symtripal Liver Type Dry Flavour); colloidal silicon dioxide; and magnesium stearate.

12. The palatable veterinary dosage form of clause 11, comprising 25% to 30% (wt/wt) microcrystalline cellulose; 5% to 6% (wt/wt) lactose monohydrate; 0.05% to 0.06% (wt/wt) butylated hydroxytoluene; 3% to 5% (wt/wt) croscarmellose sodium; 0.4% to 0.5% (wt/wt) sodium lauryl sulfate; 1% to 3% (wt/wt) povidone K30; 1% to 2% (wt/wt) Eudragit RL 30D; 0.1% to 0.5% (wt/wt) Plasacryl HTP20; 5% to 20% of a flavorant (e.g., Dtech 8P0910748 Veggie, or Symtripal Liver Type Dry Flavour); 0.5% to 1.5% (wt/wt) colloidal silicon dioxide; and 0.5% to 1.5% (wt/wt) magnesium stearate.

13. The palatable veterinary dosage form of any one of clauses 1-12, produced by a process comprising providing a first granulate comprising praziquantel and optionally moxidectin; optionally coating the first granulate with a physiologically acceptable polymer matrix (e.g., a polymethacrylate-based copolymer matrix) to provide a coated first granulate; providing a second granulate comprising the isoxazoline class parasiticide; combining the first granulate or coated first granulate with the second granulate and at least one pharmaceutically acceptable excipient to provide a third granulate; and compressing the third granulate into a tablet.

14. The palatable veterinary dosage form of clause 13, wherein the first granulate or coated first granulate comprises praziquantel, moxidectin, a diluent, an antioxidant, a disintegrant, a wetting agent, and a binder; the physiologically acceptable polymer matrix comprises a polymethacrylate-based copolymer; the second granulate comprises lotilaner, a diluent, a wetting agent, a disintegrant, and a binder; and the at least one pharmaceutically acceptable excipient comprises a diluent, a flavoring agent, a disintegrant, a glidant, and a lubricant.

15. The palatable veterinary dosage form of clause 14, wherein the first granulate or coated first granulate comprises praziquantel, moxidectin, lactose monohydrate, microcrystalline cellulose, butylated hydroxytoluene, croscarmellose sodium, sodium lauryl sulfate, and povidone K30; the physiologically acceptable polymer matrix comprises Eudragit RL 30D and Plasacryl HTP20; the second granulate comprises lotilaner, microcrystalline cellulose, sodium lauryl sulfate, croscarmellose sodium, and povidone K30; and the at least one pharmaceutically acceptable excipient comprises microcrystalline cellulose, a flavoring agent, croscarmellose sodium, colloidal silicone dioxide, and magnesium stearate.

16. The palatable veterinary dosage form of any one of clauses 13-15, wherein the granulates are substantially maintained in granular form during the compression.

17. A method of treating a parasitic infection, comprising administering to a non-human mammal in need thereof a palatable veterinary dosage form providing a dosage of about 20 mg/kg lotilaner, about 0.01to about 0.05 mg/kg moxidectin, and about 5 mg/kg praziquantel, wherein the praziquantel, and optionally moxidectin, are confined to discrete areas within the dosage form.

18. The method of clause 17, wherein the palatable veterinary dosage form further provides about 5 mg/kg pyrantel or a salt thereof.

19. The method of clause 17 or clause 18, wherein the palatable veterinary dosage form provides 0.01 to 0.02 mg/kg moxidectin.

20. The method of any one of clauses 17-19, wherein the palatable veterinary dosage form is a hard chewable tablet.

21. A process for making a palatable veterinary tablet, comprising:
providing a first granulate comprising praziquantel and optionally moxidectin;
optionally coating the first granulate with a physiologically acceptable polymer matrix (e.g., a polymethacrylate-based copolymer matrix) to provide a coated first granulate;
providing a second granulate comprising an isoxazoline class parasiticide;
combining the first granulate or coated first granulate with the second granulate and at least one pharmaceutically acceptable excipient to provide a third granulate; and
compressing the third granulate into a tablet.

22. The process of clause 21, wherein the granulates are substantially maintained in granular form during the compression.

23. The process of clause 21 or 22, wherein the at least one pharmaceutically acceptable excipient comprises at least 15%, preferably about 15-30%, microcrystalline cellulose.

24. The process of any one of clauses 21-23, wherein the second granulate further comprises pyrantel or a salt thereof.

25. The process of any one of clauses 21-24, wherein coating the first granulate with a physiologically acceptable polymer matrix (e.g., a polymethacrylate-based copolymer matrix) is conducted with a fluidized bed reactor configured for top-spray coating.

26. The process of any one of clauses 21-24, wherein coating the first granulate with a physiologically acceptable polymer matrix (e.g., a polymethacrylate-based copolymer matrix) is conducted with a fluidized bed reactor configured for bottom-spray coating.

27. The process of any one of clauses 21-24, wherein coating the first granulate with a physiologically acceptable polymer matrix (e.g., a polymethacrylate-based copolymer matrix) is conducted with a fluidized bed reactor configured for tangential-spray coating.

28. A palatable veterinary dosage form comprising
an isoxazoline class parasiticide (e.g., lotilaner, sarolaner, afoxolaner, fluralaner);
a macrocyclic lactone class parasiticide (e.g. milbemycin oxime, moxidectin, doramectin selametin); and a pyrazinoisoquinoline class parasiticide (e.g. praziquantel),
wherein pyrazinoisoquinoline class parasiticide, preferably praziquantel, and optionally the macrocyclic lactone class parasiticide, preferably moxidectin, are confined to discrete areas within the dosage form.

29. The palatable veterinary dosage form of clause 28, further comprising a tretrahydropyrimidine class parasiticide, preferably pyrantel or a salt thereof.

30. The palatable veterinary dosage form of clause 28 or clause 29, wherein the moxidectin and praziquantel are comprised within physiologically acceptable polymer matrix-coated granules (e.g., polymethacrylate-based copolymer-coated granules).

31. The palatable veterinary dosage form of any one of clauses 28-30, wherein the palatable veterinary dosage form is a hard chewable tablet.

32. The palatable veterinary dosage form of any one of clauses 28-31, wherein the palatable veterinary dosage form disintegrates in less than or equal to 15 minutes in water at 37° C., as determined by the disintegration method 2.9.1 (Test A and B) of the European Pharmacopoeia 8.0.

33. The palatable veterinary dosage form of any one of clauses 28-32, wherein the palatable veterinary dosage form has a friability of 1% or less as measured according to USP <1216>.

34. The palatable veterinary dosage form of any one of clauses 28-33, comprising 20-40% (wt/wt) lotilaner, 0.020% to 0.045% (wt/wt) moxidectin, and 5-10% (wt/wt) praziquantel, based on total weight of the dosage form.

35. The palatable veterinary dosage form of any one of clauses 28-34, further comprising at least one of a diluent (e.g, microcrystalline cellulose); an antioxidant (e.g., butylated hydroxytoluene); a disintegrant (e.g., croscarmellose sodium); a wetting agent (e.g., sodium lauryl sulfate); a binder (e.g., povidone 1K30); a taste-masking polymer (e.g., Eudragit RL 30D); a plasticizer (e.g., Plasacryl HTP20); a flavoring agent (e.g., Dtech 8P0910748 Veggie, Symtripal Liver Type Dry Flavour); a glidant (e.g., colloidal silicon dioxide); or a lubricant (e.g., magnesium stearate).

36. The palatable veterinary dosage form of clause 35, comprising microcrystalline cellulose; lactose monohydrate; butylated hydroxytoluene; croscarmellose sodium; sodium lauryl sulfate; povidone K30; Eudragit RL 30D; Plasacryl HTP20; a flavorant (e.g., Dtech 8P0910748 Veggie, and/or Symtripal Liver Type Dry Flavour); colloidal silicon dioxide; and magnesium stearate.

37. The palatable veterinary dosage form of clause 36, comprising 25% to 30% (wt/wt) microcrystalline cellulose; 3% to 6% (wt/wt) lactose monohydrate; 0.05% to 0.06% (wt/wt) butylated hydroxytoluene; 3% to 5% (wt/wt) croscarmellose sodium; 0.4% to 0.6% (wt/wt) sodium lauryl sulfate; 1% to 3% (wt/wt) povidone K30; 1% to 2% (wt/wt) Eudragit RL; 0.1% to 0.5% (wt/wt) Plasacryl HTP20; 5% to 20% of a flavorant (e.g., Dtech 8P0910748 Veggie, and/or Symtripal Liver Type Dry Flavour); 0.5% to 1.5% (wt/wt) colloidal silicon dioxide; and 0.5% to 1.5% (wt/wt) magnesium stearate.

38. The palatable veterinary dosage form of any one of clauses 28-37, produced by a process comprising providing a first granulate comprising praziquantel and optionally moxidectin; optionally coating the first granulate with a physiologically acceptable polymer matrix (e.g., a polymethacrylate-based copolymer matrix) to provide a coated first granulate; providing a second granulate comprising the isoxazoline class parasiticide; combining the first granulate or coated first granulate with the second granulate and at least one pharmaceutically acceptable excipient to provide a final blend; and compressing the ready-to-press mixture into a tablet.

39. The palatable veterinary dosage form of clause 38, wherein the first granulate or coated first granulate comprises praziquantel, moxidectin, a diluent, an antioxidant, a disintegrant, a wetting agent, and a binder; the physiologically acceptable polymer matrix comprises a polymethacrylate-based copolymer; the second granulate comprises lotilaner, a diluent, a wetting agent, a disintegrant, and a binder; and the at least one pharmaceutically acceptable excipient comprises a diluent, a flavoring agent, a disintegrant, a glidant, and a lubricant.

40. The palatable veterinary dosage form of clause 39, wherein the first granulate or coated first granulate comprises praziquantel, moxidectin, lactose monohydrate, microcrystalline cellulose, butylated hydroxytoluene, croscarmellose sodium, sodium lauryl sulfate, and povidone K30; the physiologically acceptable polymer matrix comprises Eudragit RL 30D and Plasacryl HTP20; the second granulate comprises lotilaner, microcrystalline cellulose, sodium lauryl sulfate, croscarmellose sodium, and povidone K30; and the at least one pharmaceutically acceptable excipient comprises microcrystalline cellulose, a flavoring agent, croscarmellose sodium, colloidal silicone dioxide, and magnesium stearate.

41. The palatable veterinary dosage form of clauses 38 to 40, wherein the second granulate additionally comprises a tetrahydropyrimidine class parasiticide, preferably pyrantel or a salt thereof, more preferably pyrantel pamoate.

42. The palatable veterinary dosage form of any one of clauses 38-41, wherein the granulates are substantially maintained in granular form during the compression.

43. A method of treating a parasitic infection, comprising administering to a non-human mammal in need thereof a palatable veterinary dosage form providing a dosage of about 20 mg/kg lotilaner, about 0.01 to about 0.05 mg/kg moxidectin, and about 5 mg/kg praziquantel, wherein the praziquantel, and optionally moxidectin, are confined to discrete areas within the dosage form.

44. The palatable veterinary dosage form of any one of clauses 28-42 for use in the control of parasites in and/or on animals.

45. The method of clause 43, wherein the palatable veterinary dosage form further provides about 5 mg/kg pyrantel or a salt thereof.

46. The method of clause 43 or clause 45, wherein the palatable veterinary dosage form provides 0.01 to 0.02 mg/kg moxidectin.

47. A process for making a palatable veterinary tablet, comprising:
providing a first granulate comprising praziquantel and optionally moxidectin;
optionally coating the first granulate with a physiologically acceptable polymer matrix (e.g., a polymethacrylate-based copolymer matrix) to provide a coated first granulate;
providing a second granulate comprising an isoxazoline class parasiticide;
combining the first granulate or coated first granulate with the second granulate and at least one pharmaceutically acceptable excipient to provide a final blend; and compressing the ready-to-press mixture into a tablet.

48. The process of clause 47, wherein the granulates are substantially maintained in granular form during the compression.

49. The process of clause 47 or 48, wherein the at least one pharmaceutically acceptable excipient comprises at least 15%, preferably about 15-30%, microcrystalline cellulose.

50. The process of clauses 47-49, wherein the second granulate further comprises pyrantel or a salt thereof.

51. The process of any one of clauses 47-50, wherein coating the first granulate with a physiologically acceptable polymer matrix (e.g., a polymethacrylate-based copolymer matrix) is conducted with a fluidized bed reactor configured for top-spray coating.

52. The process of any one of clauses 47-50, wherein coating the first granulate with a physiologically acceptable polymer matrix (e.g., a polymethacrylate-based copolymer matrix) is conducted with a fluidized bed reactor configured for bottom-spray coating.

### 6. EXAMPLES

The following examples are provided to illustrate the invention and are not intended to be limiting in any way.

### Example 1

### Lotilaner/Moxidectin/Praziquantel Tablet

Table 1 shows a tablet composition prepared with lotilaner, moxidectin, and praziquantel as active pharmaceutical ingredients. Table 2 provides exemplary process parameters consistent with those used to produce the praziquantel/moxi dectin granulation and coating of Table 1. Lotilaner was granulated along with the identified excipients in a high shear granulator, dried, and sized. Praziquantel and moxidectin were granulated in a high shear granulator, dried and sized. Sized praziquantel and moxidectin granules were coated with Eudragit RL 30D in a fluidized bed coater (top or bottom spray) to a predetermined coating level. Coated granules were sieved. Lotilaner and coated praziquantel and moxidectin granules were blended along with extra granular excipients and lubricated for compression. The lubricated blend was compressed on a compression machine per weight of the selected drug product parameters, as shown in Table 3. The tablets were light brown colored, round biconvex tablets with brownish spots without embossing for oral use. Tablets were packaged in blister packaging.

TABLE 1
Tablet Composition

| | | | | Tablet |
|---|---|---|---|---|
| | | | | Composition |
| Unit of Operation | | Ingredient | Function | (% w/w) |
| Moxidectin | (1.97 mg/g)/ | Moxidectin | API | 0.036 |
| Praziquantel | (500 mg/g) | Praziquantel | API | 9.120 |
| Granulation | | Lactose Monohydrate (Granulac 200) | Diluent | 5.472 |
| | | Microcrystalline Cellulose (Type 101) | Diluent | 2.077 |
| | | Butylated Hydroxytoluene | Antioxidant | 0.055 |
| | | Croscarmellose Sodium | Disintegrant | 0.912 |
| | | Sodium Lauryl Sulfate | Wetting Agent | 0.021 |
| | | Povidone K30 | Binder | 0.547 |
| | | Purified Water | Process aid | — |
| Moxidectin | (1.79 mg/g)/ | Eudragrit RL 30D | Taste masking | 1.330 |
| Praziquantel | (454.5 mg/g) | | polymer | |
| Taste Masking Granules | | Plasacryl HTP20 | Plasticizer for | 0.266 |
| | | | Eudragrit RL | |
| | | | 30D | |
| | | Purified Water | Process aid | — |
| Lotilaner | (800 mg/g) | Lotilaner | API | 36.000 |

| | | | |
|---|---|---|---|
| Granulation | Microcrystalline Cellulose (Type 101) | Diluent | 6.600 |
| | Sodium Lauryl Sulfate | Wetting Agent | 0.450 |
| | Croscarmellose Sodium | Disintegrant | 0.600 |
| | Povidone K30 | Binder | 1.350 |
| | Purified Water | Process aid | — |
| Dry Blend | Microcrystalline Cellulose (Avicel 200) | Diluent | 18.664 |
| | Dtech 8P0910748 Veggie | Flavoring agent | 5.000 |
| | Symtripal Liver Type Dry Flavour | Flavoring agent | 7.500 |
| | Croscarmellose Sodium | Disintegrant | 2.000 |
| | Colloidal Silicon Dioxide | Glidant | 1.000 |
| | Magnesium Stearate | Lubricant | 1.000 |
| | Total | | 100.00 |

TABLE 2
Process Parameters For Praziquantel/Moxidectin Granulation & Coating

| Granulation: | 16.3 | kg batch size |
|---|---|---|
| Dry Pre-Blend (praxi + moxi) | 2 | min at very low speed as pre-blending |
| Dry Blend | 2 | min at medium speed as blending |
| water adding | 4 | min at high speed |
| wet massing | at high speed for 0.5-1.0 | min |
| wet sieving | at low speed with 3 | mm sieve size (round) |
| Drying: | 16.3 | kg batch size |
| pre heating | up to | 300 m³/h |
| | at 75° | C. |
| inlet air volume | up to | 300 m³/h |
| inlet temp. | 75° | C. |
| prod. temp. endpoint | 35° | C. |
| dry sieving | at low speed with | 1.0 mm sieve size |
| Loss on drying (LOD) target | ≤2.0% | |
| | (at 100° | C. criteria 3 |
| | [1 | mg/50 s]) |
| Coating (BOTTOM spray): | 6 | kg batch size |
| pre heating I | 115 | m3/h at |
| | 35° C. | without material |
| pre heating II | 115 | m3/h at |
| | 50° | C. with material |
| coating inlet air volume | 115 | m3/h |
| inlet temp. | 50° | C. |
| prod temp Coating | 30° | C. or less |
| atomization pressure | 1.8 | bar |
| spray rate | 20 | g/min or less |
| prod. temp. Drying | 35° | C. or less |
| prod. temp. cooling | 30° | C. with |
| | 20° | C. inlet air temp. |
| LOD target | ≤2.0% | |
| | (at 100° | C. criteria 3 |
| | [1 | mg/50 s]) |
| Coating (TOP spray): | 6 | kg batch size |
| pre heating I | 150 | m3/h at |
| | 35° | C. without material |
| pre heating II | 150 | m3/h at |
| | 50° | C. with material |
| coating inlet air volume | 150 | m3/h |
| inlet temp. | 60° | C. |
| prod. temp. Coating | 30° | C. or less |
| atomization pressure | 1.2 | bar |
| spray rate | 40 | g/min or less |
| prod. temp. Drying | 35° | C. or less |
| prod. temp. cooling | 30° | C. with |
| | 20° | C. inlet air temp. |

TABLE 3
Tablet Dosage Forms by Animal Weight Band

| Animal | | | | Tablet | Tablets |
|---|---|---|---|---|---|
| Weight | Lotilaner | Moxidectin | Praziquantel | Weight | Dimensions |
| Band | (mg) | (mg) | (mg) | (mg) | (mm) |
| 4.4-6.0 lbs. | 56.25 | 0.05625 | 14.25 | 156.25 | 7.25 |
| (2-2.7 kg) | | | | | |
| 6.1-12.0 lbs. | 112.50 | 0.1125 | 28.50 | 312.50 | 9.5 |
| (2.8-5.4 kg) | | | | | |
| 12.1-25 lbs. | 225.00 | 0.225 | 57.00 | 625.00 | 12 |
| (5.5-11.25 kg) | | | | | |
| 25.1-50 lbs. | 450.00 | 0.450 | 114.00 | 1250.00 | 15.2 |
| (11.3-22.5 kg) | | | | | |
| 50.1-100 lbs. | 900.00 | 0.900 | 228.00 | 2500.00 | 19.2 |
| (22.6-45 kg) | | | | | |

### Example 2

### Lotilaner/MoxidectinPraziquantel Tablet

Tablets having the composition shown in Table 4 were prepared as described in Example 1.

TABLE 4
Tablet Composition

| | | | Tablet |
|---|---|---|---|
| | | | Composition |
| Unit of Operation | Ingredient | Function | (% w/w) |
| Moxidectin (1.97 mg/g)/ | Moxidectin | API | 0.036 |
| Praziquantel (500 mg/g) | Praziquantel | API | 9.120 |
| Granulation | Lactose Monohydrate (Granulac 200) | Diluent | 5.472 |
| | Microcrystalline Cellulose (Type 101) | Diluent | 2.077 |
| | Butylated Hydroxytoluene | Antioxidant | 0.055 |
| | Croscarmellose Sodium | Disintegrant | 0.912 |
| | Sodium Lauryl Sulfate | Wetting Agent | 0.021 |
| | Povidone K30 | Binder | 0.547 |
| | Purified Water | Process aid | — |
| Moxidectin (1.79 mg/g)/ | Eudragrit RL 30D | Taste masking | 1.520 |
| Praziquantel (454.5 mg/g) | | polymer | |
| Taste Masking Granules | Plasacryl HTP20 | Plasticizer for | 0.304 |
| | | Eudragrit RL | |
| | | 30D | |
| | Purified Water | Process aid | — |
| Lotilaner (800 mg/g) | Lotilaner | API | 36.000 |
| Granulation | Microcrystalline Cellulose (Type 101) | Diluent | 6.600 |
| | Sodium Lauryl Sulfate | Wetting Agent | 0.450 |
| | Croscarmellose Sodium | Disintegrant | 0.600 |
| | Povidone K30 | Binder | 1.350 |
| | Purified Water | Process aid | — |
| Dry Blend | Microcrystalline Cellulose (Avicel 200) | Diluent | 18.436 |
| | Dtech 8P0910748 Veggie | Flavoring agent | 5.000 |
| | Symtripal Liver Type Dry Flavour | Flavoring agent | 7.500 |
| | Croscarmellose Sodium | Disintegrant | 2.000 |
| | Colloidal Silicon Dioxide | Glidant | 1.000 |
| | Magnesium Stearate | Lubricant | 1.000 |
| | Total | | 100.00 |

### Example 3

### Lotilaner/Moxidectin/Praziquantel/Pyrantel Tablet

The composition of a Lotilaner/Moxidectin/Praziquantel/Pyrantel Pamoate tablet is given in table 5. The Moxidectin/Praziquantel granules are manufactured similarly to the above-mentioned granulation process (see in particular Example 1). Afterwards, a coating step is performed.

For the second granules, Lotilaner and Pyrantel Pamoate were granulated in a high shear granulator, dried and sieved. Both granules and the post-blend were combined under mixing. The resulting final blend was then compressed into tablets of different sizes (e.g. with a total weight of 236 mg, 472 mg, 945 mg, 1889 mg or 3778 mg per tablet). The tablets were light brown colored, round biconvex tablets with brownish spots. Tablets were packaged in blister packaging.

TABLE 5
Composition of Lotilaner/Moxidectin/Praziquantel/Pyrantel Pamoate Tablet

| | | Tablet |
|---|---|---|
| | | Composition |
| Unit of Operation | Ingredient | (% w/w) |
| Moxidectin (2.46 mg/g)/ | Moxidectin | 0.024 |
| Praziquantel (500 mg/g) | Praziquantel | 6.035 |
| Granulation | Lactose Monohydrate (Granulac 200) | 3.621 |
| | Microcrystalline Cellulose (Type 101) | 1.374 |
| | Granulact (Basel) oder Vivapur (Huningue) | |
| | Butylated Hydroxytoluene (BHT) | 0.036 |
| | Croscarmellose Sodium | 0.603 |
| | Sodium Lauryl Sulfate (SLS) | 0.014 |
| | Povidone K30 | 0.362 |
| | Purified Water* | — |
| Part of uncoated Granules in Final Blend | | 12.06882 |
| Moxidectin/Praziquantel | Eudragrit RL 30D | 1.006 |
| Granules Coated with 10% | Plasacryl HTP20 | 0.201 |
| RL30D | Purified Water* | — |
| Part of coated Granules in Final Blend | | 13.2758 |
| Lotilaner Pyrantel | Lotilaner | 23.822 |
| (800 mg/g) Granulation | Pyrantel Pamoate | 17.392 |
| | Microcrystalline Cellulose (Type 101) | 7.815 |
| | Sodium Lauryl Sulfate (SLS) | 0.518 |
| | Croscarmellose Sodium | 0.690 |
| | Povidone K30 | 1.554 |
| | Purified Water* | — |
| Part of Lotilaner Pyrantel Granules in Final Blend | | 51.7910 |
| Dry Blend | Microcrystalline Cellulose (Type 200) ** | 17.436 |
| | Dtech 8P0910748 Veggie | 5.000 |
| | Symtripal Liver Type Dry Flavour | 7.500 |
| | Croscarmellose Sodium | 3.000 |
| | Colloidal Silicon Dioxide | 1.000 |
| | Magnesium Stearate | 1.000 |
| Part of Dry Blend Granules in Final Blend | | 34.9360 |
| | Total | 100.00 |

*Evaporated during the drying process and therefore excluded from the calculation

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A method of treating a parasitic infection, comprising administering to a non-human mammal in need thereof a palatable veterinary dosage form comprising
an isoxazoline class parasiticide selected from the group consisting of lotilaner, sarolaner, afoxolaner, and fluralaner;
and a first granulate comprising granules, wherein the granules of the first granulate comprise
a macrocyclic lactone class parasiticide; and
a pyrazinoisoquinoline class parasiticide.

2. The method of claim 1, wherein the pyrazinoisoquinoline class parasiticide comprises praziquantel.

3. The method of claim 1, wherein the macrocyclic lactone class parasiticide comprises moxidectin.

4. The method of claim 1, wherein the first granulate comprises physiologically acceptable polymer matrix-coated granules.

5. The method of claim 1, wherein the dosage form comprises 20-40% (wt/wt) lotilaner, 0.020% to 0.045% (wt/wt) moxidectin, and 5-10% (wt/wt) praziquantel, based on total weight of the dosage form.

6. The method of claim 1, wherein the dosage form comprises at least one of a diluent; an antioxidant; a disintegrant; a wetting agent; a binder; a taste-masking polymer; a plasticizer; a flavoring agent; a glidant; or a lubricant.

7. The method of claim 1, wherein the dosage form comprises microcrystalline cellulose; lactose monohydrate; butylated hydroxytoluene; croscarmellose sodium; sodium lauryl sulfate; polyvinylpyrrolidone; ammonia methacrylate copolymer type A; a 20% aqueous emulsion comprising glycerol monostearate, triethyl citrate, polysorbate 80; a flavorant; colloidal silicon dioxide; and magnesium stearate.

8. The method of claim 1, comprising administering to a non-human mammal in need thereof a palatable veterinary dosage form providing a dosage of about 20 mg/kg lotilaner, about 0.01 to about 0.05 mg/kg moxidectin, and about 5 mg/kg praziquantel, wherein the praziquantel, and moxidectin, are confined to discrete areas within the dosage form.

9. The method of claim 1, wherein the palatable veterinary dosage form further provides about 5 mg/kg pyrantel or a salt thereof.

10. The method of claim 1, wherein the palatable veterinary dosage form provides 0.01 to 0.02 mg/kg moxidectin.

11. The method of claim 1, wherein the palatable veterinary dosage form is a hard chewable tablet.

12. The method of claim 1, wherein the administration is monthly.

13. The method of claim 1, wherein the non-human mammal is a cat or a dog.

14. The method of claim 13, wherein the non-human mammal is a dog.

15. A process for making a palatable veterinary tablet, comprising providing a first granulate comprising praziquantel and optionally moxidectin; providing a second granulate comprising an isoxazoline class parasiticide; combining the first granulate with the second granulate and at least one pharmaceutically acceptable excipient to provide a third granulate; and compressing the third granulate into a tablet.

16. The process of claim 15, further comprising coating the first granulate with a physiologically acceptable polymer matrix optionally a polymethacrylate-based copolymer matrix to provide a coated first granulate; combining the coated first granulate with the second granulate and at least one pharmaceutically acceptable excipient to provide a third granulate; and compressing the third granulate into a tablet.

17. The process of claim 15, wherein the second granulate further comprises pyrantel or a salt thereof.

18. The process of claim 15, wherein the granulates are substantially maintained in granular form during the compression.

19. The process of claim 15, wherein the at least one pharmaceutically acceptable excipient comprises at least 15% microcrystalline cellulose.

20. The process of claim 15, wherein coating the first granulate with a physiologically acceptable polymer matrix is conducted with a fluidized bed reactor configured for top-spray coating, bottom-spray coating, or tangential-spray coating.
